(19) United States
(12) Patent Application Publication
(10) Pub. No.: US 2004/0210312 A1
(43) Pub. Date: Oct. 21, 2004

(54) Implant for insertion between vertebrae of the spinal column

(76) Inventor: Carsten Neumann, Bad Abbach (DE)

Correspondence Address: COLLARD & ROE, P.C., 1077 Northern Boulevard, Roslyn, NY, 11576-1696, US

(21) Appl. No.: 10/801,119

(22) Filed: Mar. 15, 2004

(30) Foreign Application Priority Data
Mar. 15, 2003 (DE) 103 11 477.7

(51) Int. Cl. A61F 2/44

(52) U.S. Cl. 623/17.11; 623/17.16

(57) ABSTRACT

An implant for insertion between vertebrae of the spinal column, as a substitute for disks, vertebrae, or parts of vertebrae removed from the spinal column. The implant has a first implant part and a second implant part, which are adjustable, relative to one another, in the direction of their coaxial longitudinal axes. The implant also has a joining plate that can be connected with the free end of at least one of the two implant parts, in an essentially perpendicular alignment relative to the longitudinal axis, by means for releasable attachment. This adjustability is to change the length of the implant.

### CROSS REFERENCE TO RELATED APPLICATIONS

Applicant claims priority under 35 U.S.C. §119 of German Application No. 10 311 477.7 filed Mar. 15, 2003.

### BACKGROUND OF THE INVENTION

1. Field of the Invention

The present invention relates to an implant for insertion between vertebrae of the spinal column, as a substitute for disks, vertebrae, or parts of vertebrae removed from the spinal column. This device has a first implant part and a second implant part, which are adjustable, relative to one another, along their coaxial longitudinal axes, to change a length of the implant.

2. The Prior Art

An implant of this type is known, for example, from DE 44 23 2057 A1. This type of implant has proven itself in practice and has the simple possibility of distraction inherent in it, such that the two end implant parts that are arranged on the center implant part are screwed into the center implant part by way of their threaded connection and thereby are adjusted in height in the manner of a spindle. However, there must be must be a relatively great space requirement that is needed for the distraction, to be able to pivot a tool during the operation and thereby to rotate the implant parts relative to one another.

### SUMMARY OF THE INVENTION

One of the goals of the invention is to make possible an individual adjustment of the implant to the conditions prevailing in situ. This design simplifies the inventory by making different size implants available, and also for facilitating the distraction that is to be performed during the operation.

This task is accomplished, by means of an implant of the type stated initially, which has a joining plate that can be connected with the free end of at least one of two implant parts. This connection is in an essentially perpendicular alignment relative to a longitudinal axis, wherein a joining plate is coupled by means of a releasable attachment.

One of the benefits of this implant is that the length expanse of the implant can be changed in a simple-manner, by means of a joining plate. Thus, the implant itself can already be made available in a less precise gradation. Furthermore, this joining plate allows for an enlargement of the range that can be covered with regard to the desired length change of the distraction. Thus, the extent of the distraction with the required reciprocal adjustment of the implant parts is reduced by the thickness of the joining plate.

Thus, it is advantageous if the thickness of the joining plate corresponds to a range between 2.0% to 30.0% of the height of one of the implant parts.

In a preferred embodiment of the invention the joining plate projects beyond an outside contour of the implant. This connection provides the advantage such that the effective active cross-section of the implant is no longer solely determined by its outside contour, but rather it can be adapted for variation of the surface pressure, via a suitable selection of the size of the joining plate.

For simple handling during an operation, the connection of the joining plate with the implant should be produced and released again in simple manner. Thus, the means for releasable attachment may comprise an opening formed in the joining plate, so that a plug-in connection can be produced in the simplest possible manner, by means of setting the joining plate onto an implant with its opening. Thus, the joining plate is preferably set onto the implant on the outside, so that the shape of the opening is adapted to the outside contour of an implant.

In an alternative embodiment of the invention, the opening may be configured in the center of gravity of the joining plate, to obtain a symmetrical configuration with reference to the longitudinal axis of the implant.

However, it is a also possible to configure the opening to lie outside of the center of gravity of the joining plate. In this case, it is then suitable for situations in which a support surface of the vertebrae associated with the implant can be enlarged on one side. Thus, the implant can be used in twos, in pairs, to bridge the spinal column gap between two vertebrae.

There is an improved contact surface between the implant and the vertebrae by creating a surface of the joining plate that faces the vertebrae in a domed or convex manner.

The joining plate can have a means for attachment in duplicate which is assigned to one of the implant parts, in each instance. Thus, the advantages of the invention can be utilized for every vertebrae that is adjacent to the implant.

To make the handling of the implant having the joining plate as simple as possible under the conditions of an operation, the means for attachment are formed by a plug-in connection that is formed between the joining plate and the implant part and a catch seat. This is possible, for example, such that the means for attachment are formed by a bayonet closure. Alternatively, the means for attachment can comprise a groove formed in the free end of the implant part, as well as a spring mounted in a groove of the opening of the joining plate. This spring which moves back, in a resilient manner, when the joining plate is set onto the implant part, can enter into the groove of the implant part as a catch member.

To facilitate reliable anchoring of the implant in the vertebrae, the mandrels and/or cutting blades can be arranged on the side of the joining plate that faces the vertebrae.

The implant, structured as a modular system, can be made variable in shape wherein the joining plate is configured as a polygon, in a rounded manner, or, alternatively, in a star-shaped manner. The star-shaped configuration of the joining plate, in particular, is set so that a closed surface is formed as a sharp separating plane between the vertebrae and the implant arranged below the joining plate. This creates the possibility that an osseous connection will form between the two vertebrae wherein an implant can grow into place particularly well, particularly if bone chips, bone cement, or the like is used in supportive manner.

The advantage cited above not only exists if the joining plate is configured in star-shaped manner, but also if a passage opening is formed in the joining plate. To facilitate particularly good osseous penetration of the implant, it is practical if the passage opening is provided multiple times.

In addition, the passage opening may extend to an outer edge of the joining plate, since the accessibility for deposition of bone-building material is simplified, such that there is a larger contact surface, or a length for deposition of this material.

In another embodiment of the invention, plate ridges proceed from the opening form and delimit the passage opening. The stability of a joining plate is configured such that it is improved so that the plate ridges are connected with one another at their free ends, to form the edge of the joining plate.

In another preferred embodiment of the invention, a rotatable threaded ring is assigned to the first implant part, which engages with a ring thread in a thread assigned to the second implant part. The threaded ring has a bevel wheel gearing. In the case of an implant configured in this manner, the space requirement for changing the length of the implant, in other words the distraction, is reduced by avoiding a pivot movement.

To adapt to the anatomical conditions, the side of the joining plate that faces the vertebrae and/or the side of the joining plate that faces the implant part is oriented at an incline to the longitudinal axis, whereby the angle of incline is between 3 and 45 degrees.

Good adaptation to the anatomy requires that the rotational position of the joining plate can be fixed in place about the longitudinal axis, relative to the implant part. Thus, for this purpose, the implant may have catch seats that are formed between the joining plate and the implant part, in the circumference direction, in step widths from 10 degrees to 45 degrees.

### BRIEF DESCRIPTION OF THE DRAWINGS

Other objects and features of the present invention will become apparent from the following detailed description considered in connection with the accompanying drawings. It should be understood, however, that the drawings are designed for purpose of illustration only and not as a definition of the limits of the invention.

FIG. 1 is a perspective view of an implant comprising a first implant part and a second implant part, having two joining plates attached at the free ends of the two implant parts;

FIG. 2 is the implant from FIG. 1 shown in a side view;

FIG. 3 is a view of an implant corresponding to FIG. 1, having joining plates attached to the two implant parts which are not at their centers of gravity;

FIG. 4 is a view corresponding to FIG. 2, of the implant of FIG. 3;

FIG. 5 is a view from the direction of the arrow V from FIG. 4;

FIG. 6 is a top view of the implant according to FIG. 3;

FIG. 7 is a view of another alternative embodiment, corresponding to FIG. 1;

FIG. 8 is a side view of the implant from FIG. 7;

FIG. 9 is a view of another alternative embodiment, corresponding to FIG. 1;

FIG. 10 is an isolated perspective view of a joining plate;

FIG. 11 is a top view of another joining plate;

FIG. 12 is an isolated perspective view of another joining plate;

FIG. 13 is an isolated perspective view of another joining plate;

FIG. 14 is a top view of another joining plate;

FIG. 15 is a top view of another joining plate;

FIG. 16 is an isolated perspective view of another joining plate having plate ridges;

FIG. 17 is a top view of another joining plate having plate ridges;

FIG. 18 is a view of another embodiment of a joining plate, corresponding to FIG. 17;

FIG. 19 is a view of another embodiment of a joining plate, corresponding to FIG. 17;

FIG. 20 is an isolated perspective view of another joining plate;

FIG. 21 is a view of another embodiment of a joining plate, corresponding to FIG. 20;

FIG. 22 is a view of another embodiment of a joining plate, corresponding to FIG. 20;

FIG. 23 is an isolated perspective view of a joining plate adapted to the outside contour of the implant;

FIG. 24 is a side view of the joining plate from FIG. 23;

FIG. 25 is a top view of the joining plate according to FIG. 23;

FIG. 26 is a view of a joining plate having non-plane-parallel surfaces, corresponding to FIG. 22;

FIG. 27 is a side view of the joining plate from FIG. 26;

FIG. 28 is a top view of a joining plate;

FIG. 29 is the section XXIX-XXIX from FIG. 28;

FIG. 30 is a side view of the joining plate from FIG. 20; and

FIG. 31 is a view of an implant having two joining plates according to FIG. 28, corresponding to FIG. 1.

### DETAILED DESCRIPTION OF PREFERRED EMBODIMENTS

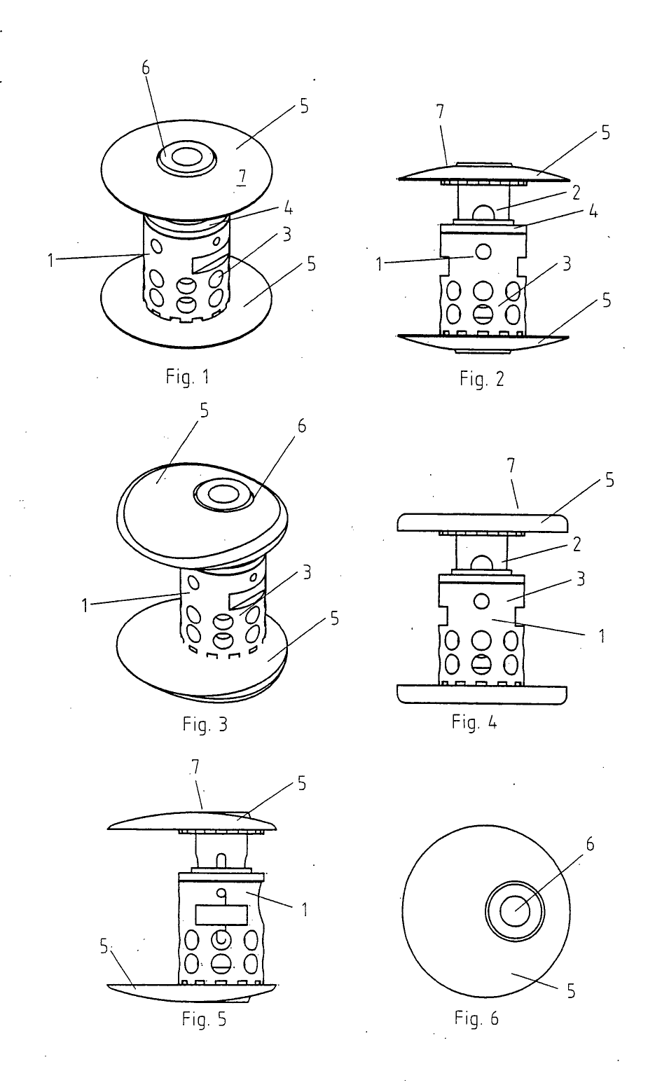

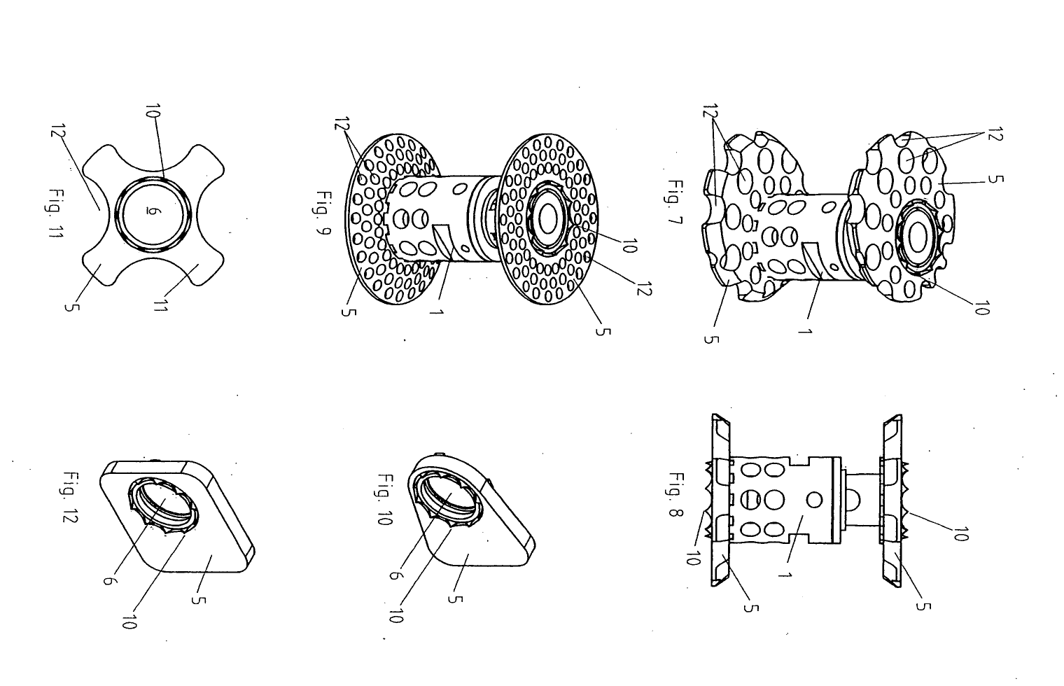

Referring to the drawings FIGS. 1 and 2 show an implant 1 that can be inserted between vertebrae of the spinal column, (not shown), to substitute for disks, vertebrae, or parts of vertebrae removed from the spinal column. Implant 1 comprises a first implant part 2 and a second implant part 3, which are axially adjustable, relative to one another, along their longitudinal axis, to change the length of implant 1. FIGS. 1 and 2 show a rotatable threaded ring 4 that is coupled to or assigned to second implant part 3, which engages with a ring thread in a thread assigned to first implant part 2. Threaded ring 4 has a bevel wheel gearing, so that a second bevel wheel, set against the bevel wheel gearing can bring about a change in length of implant 1.

Implant 1 furthermore has at least one joining plate 5. A total of two joining plates 5 that can be connected with the free end of the two implant parts 2, 3 is shown in the exemplary embodiment. The connection can be in an essentially perpendicular alignment relative to the longitudinal axis, by means of a releasable attachment. Joining plate 5 has a means for attachment in duplicate that is on either side, and in each instance is assigned to one of implant parts 2, 3. The thickness of the joining plate 5 corresponds to 2.0% to 30.0% of the height of one of these implant parts 2, 3, so that a change in the length of the implant 1 can essentially be brought about by joining plate 5, particularly by the one shown in isolated manner in FIGS. 23 to 24. The other joining plates 5 shown in the drawing project beyond the outside contour of implant 1 and thereby make available a larger contact surface relative to the adjacent vertebrae for implant 1.

The means for releasable attachment comprises an opening 6 formed in joining plate 5, which is adapted to the outside contour of implant 1, so that joining plate 5 can be set onto implant 1 in the simplest possible manner. With the embodiment shown in FIGS. 1 and 2, opening 6 is disposed configured in the center of gravity of joining plate 5, while FIGS. 3 to 6 show an embodiment in which opening 6 is configured outside of the center of gravity of joining plate 5.

In this regard, FIGS. 2 and 5 show that surface 7 of joining plate 5 that faces the vertebrae may be domed in convex manner.

FIGS. 23 to 25 show that the plug-in connection may also have a catch seat, specifically formed, in concrete terms, via a groove formed at a free end of implant part 2, 3, and also by a spring 9 mounted in a groove 8 of opening 6 of joining plate 5. FIG. 24, in particular, shows that mandrels 10 may be arranged on a side of joining plate 5 that faces the vertebrae, which can also be configured as cutting blades with a longer expanse in the circumferential direction.

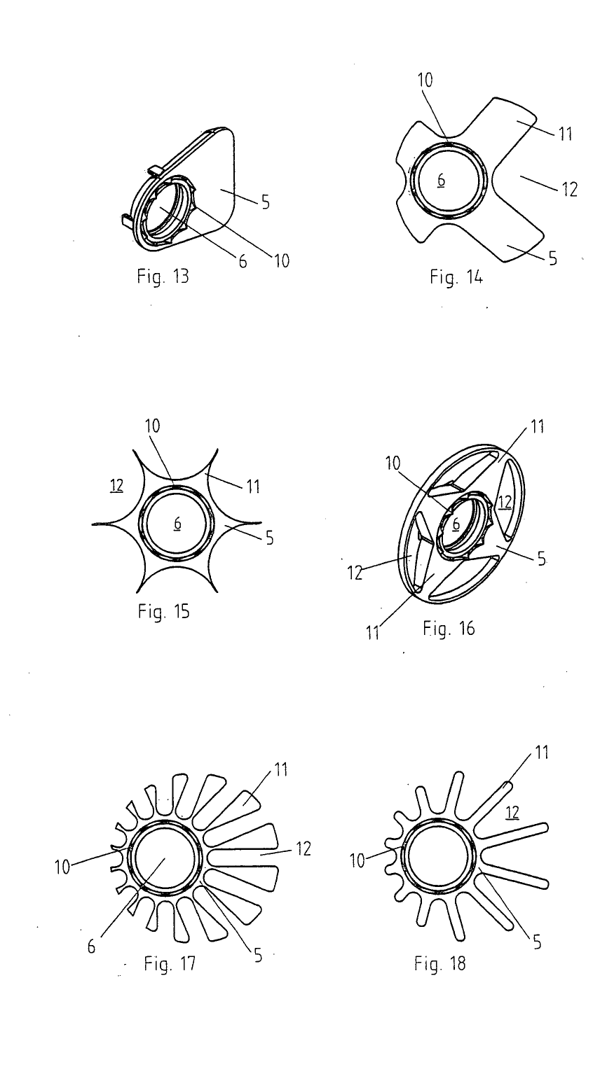

Joining plate 5, is configured so that there are many different possibilities of variation to take the actual conditions present in a particular operation into account. FIG. 10, similar to FIG. 13, shows an essentially triangular configuration of joining plate 5, with rounded corners, while FIG. 12 shows a rectangular configuration. In this case, any polygon can be implemented, to the extent that this is desirable for anatomical or operative reasons.

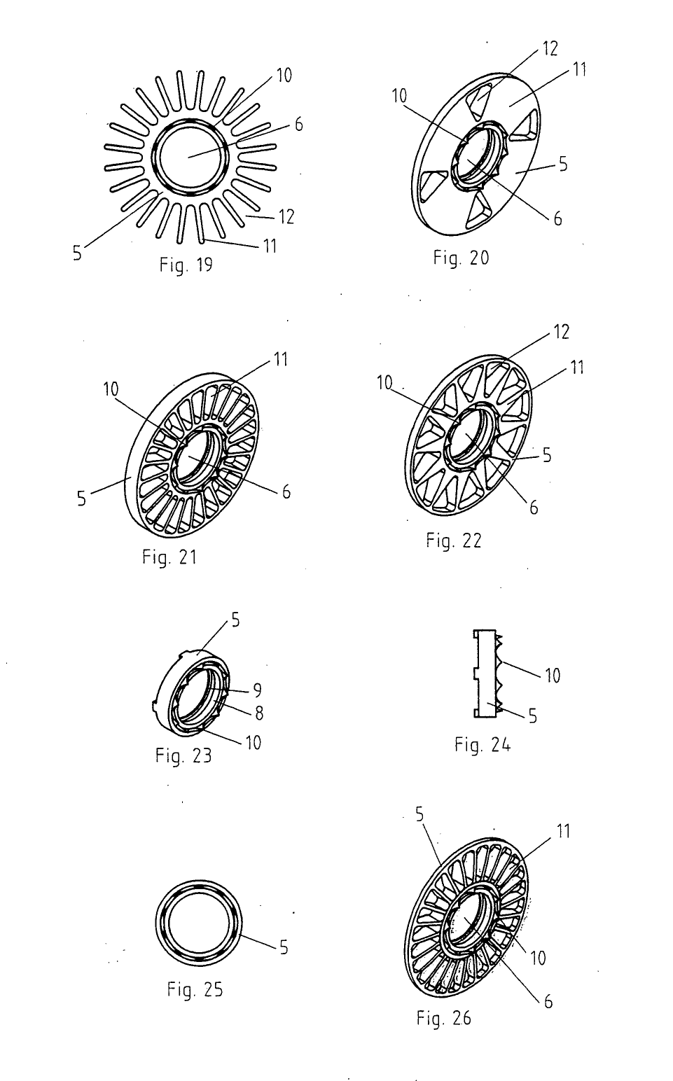

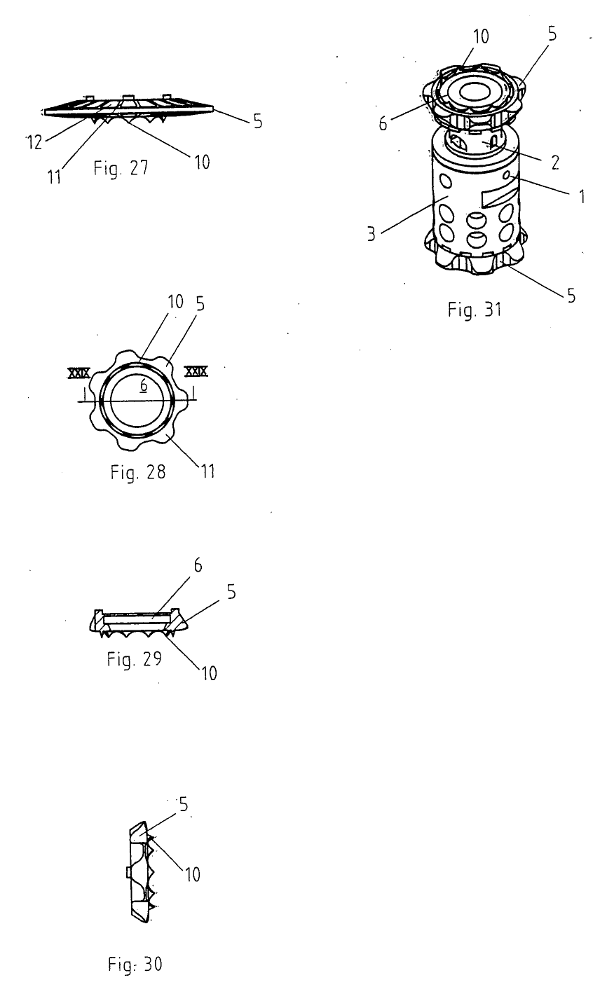

FIG. 11, similar to FIGS. 14 and 15, shows a star-shaped configuration of joining plate 5, wherein the regions between plate ridges 11 can be interpreted as passage openings 12, which allow a great approximation to the outside contour of implant 1 with bone cement, despite the given radial expanse of plate ridges 11. In total, the varied configuration of joining plate 5 also provide better support of the vertebrae, by making large-area contact possible. In this way, the majority of passage openings 12 that are shown embedded in FIG. 7 extend to the edge of joining plate 5 in FIG. 11. The embodiments shown in FIGS. 17 to 22 can be described, in the simplest manner, via plate ridges 11 that proceed from opening 6 of joining plate 5, which form and delimit the passage openings, whereby in FIGS. 21 and 22, the free ends of plate ridges 11 are connected to each other, to form an edge of joining plate 5.

FIGS. 26 and 27 show that the side of joining plate 5 that faces the vertebrae and the side of joining plate 5 that faces implant part 2, 3 are oriented at an incline to the longitudinal axis, so that implant 1 can be better adapted to the anatomical conditions found. For this purpose, joining plates 5 having an incline angle between 3° and 45° may be kept on hand.

To permanently assure the good adaptation of the implant, the rotational position of joining plate 5 can be fixed in place about the longitudinal axis, relative to implant parts 2, or 3, for which purpose catch seats may be formed between joining plate 5 and implant part 2, 3, in the circumference direction, in step widths from 10 degrees to 45 degrees.

Accordingly, while a few embodiments of the present invention have been shown and described, it is to be understood that many changes and modifications may be made thereunto without departing from the spirit and scope of the invention as defined in the appended claims.

What is claimed is:

1. An implant for insertion between vertebrae of a spinal column comprising:
a) at least two implant parts comprising:
i) a first implant part; and
ii) a second implant part wherein said first and said second implant parts are axially adjustable relative to each other to change a length of the implant along a longitudinal axis of the implant; and
b) a joining plate that can be releasably connected with a free end of at least one of said at least two implant parts in a substantially perpendicular alignment relative to said longitudinal axis of the implant, wherein the implant can be inserted between vertebrae of a spinal column as a substitute for disks, vertebrae or parts of vertebrae removed from a spinal column.

2. The implant as in claim 1, wherein said joining plate has a thickness that corresponds to between 2% and 30% of a height of at least one of said at least two implant parts.

3. The implant as in claim 1, wherein said joining plate projects beyond an outside contour of said implant.

4. The implant as in claim 1, further comprising a means for releasable attachment of said joining plate comprising an opening formed in said joining plate.

5. The implant as in claim 4, wherein a shape of said opening is adapted to an outside contour of said implant.

6. The implant as in claim 4, wherein said opening is positioned to be in a center of gravity of said joining plate.

7. The implant as in claim 4, wherein said opening is configured to lie outside of a center of gravity of said joining plate.

8. The implant as in claim 1, wherein a surface of said joining plate that faces a vertebrae is shaped as a convex dome.

9. The implant as in claim 1, wherein said joining plate has a means for attachment in duplicate and is attached to at least one of said at least two implant parts.

10. The implant as in claim 9, further comprising a catch seat wherein said means for attachment is formed by a plug-in connection formed between said joining plate and at least one of said at least two implant parts.

11. The implant as in claim 10, wherein said means for attachment is formed by a bayonet closure.

12. The implant as in claim 9, wherein said means for attachment comprises a groove formed in a free end of said at least one of said at least two implant part; and further comprising a spring mounted in a groove in an opening formed in said joining plate.

13. The implant as in claim 12, further comprising a plurality of mandrels or cutting blades coupled to a side of said joining plate that faces a vertebra.

14. The implant as in claim 3, wherein said joining plate is formed as a polygon.

15. The implant as in claim 3, wherein said joining plate is configured in a rounded manner.

16. The implant as in claim 3, wherein said joining plate is configured in a star-shaped manner.

17. The implant as in claim 3, wherein said joining plate has a passage opening.

18. The implant as in claim 17, wherein said joining plate has a plurality of passage openings.

19. The implant as in claim 18, wherein at least one of said plurality of passage openings extends to an outer edge of said joining plate.

20. The implant as in claim 18, wherein said joining plate has a plurality of plate ridges disposed around said plurality of passage openings on said joining plate.

21. The implant as in claim 20, wherein said plurality of plate ridges on said joining plate are coupled to each other at a free end of said plurality of plate ridges to form an edge of said joining plate.

22. The implant as in claim 21, further comprising a rotatable threaded ring coupled to said second implant part, and wherein said first implant part has threads which engage with a set of threads in said rotatable threaded ring, wherein said rotatable threaded ring has a bevel wheel gearing.

23. The implant as in claim 1, wherein said joining plate has a side that faces the vertebrae and is orientated at an incline to said longitudinal axis.

24. The implant as in claim 1, wherein said joining plate has a side that faces said at least two implant parts and is orientated at an incline to a longitudinal axis.

25. The implant as in claim 24, wherein an angle of said incline of said joining plate is between 3 degrees and 45 degrees.

26. The implant as in claim 23, wherein said joining plate has a rotational position that can be fixed in place about a longitudinal axis relative to said at least two implant parts.

27. The implant as in claim 26, further comprising a plurality of catch seats formed between said joining plate and said at least two implant parts in a circumferential direction, in step widths from between 10 degrees to 45 degrees.
